(19) United States
(12) Patent Application Publication
(10) Pub. No.: US 2007/0299335 A1
(43) Pub. Date: Dec. 27, 2007

(54) Estimation of blood input function for functional medical scans

(75) Inventors: Jerome Declerck, Oxford Oxfordshire (GB); David Schottlander, Oxfordshire (GB)

Correspondence Address: CROWELL & MORING LLP;INTELLECTUAL PROPERTY GROUP, P.O. BOX 14300, WASHINGTON, DC, 20044-4300, US

(73) Assignee: Siemens MEdical Solutions USA, Inc., Malvern, PA (US)

(21) Appl. No.: 11/806,656

(22) Filed: Jun. 1, 2007

(30) Foreign Application Priority Data
Jun. 2, 2006 (GB) 0610758.5

(51) Int. Cl. A61B 5/055

(52) U.S. Cl. 600/420

(57) ABSTRACT

A method is described for analysing the images produced by functional medical scans such as Positron Emission Tomography (PET), which method provides for an accurate estimation of Blood Input Function (BIF). An MRI scan and the functional scan is performed simultaneously and the results of the former is used to derive the BIF. The BIF so derived is then used in pharmacokinetic modelling along with the results of the functional scan.

The invention is concerned with functional medical imaging techniques such as Positron Emission Tomography and, in particular, with the estimation of Blood Input Function (BIF) necessary for the processing of images produced thereby.

Positron Emission Tomography (PET) is a nuclear medical imaging technique which provides a three-dimensional map of functional processes in the human or animal body.

A radioactive (positron emitting) tracer isotope is incorporated in a metabolically active molecule such as fluoro-deoxy-glucose (FDG, a sugar analog) which is ingested in a subject. Other radiolabelled molecules which may be used in PET include [11C]-raclopride, [11C]-carbon dioxide, [15O]-labeled water or oxygen, [13N]-ammonia. All of these can be imaged by a scanner or camera which detects and records the gamma type radiation resulting from a collision between an emitted positron and an electron in the surrounding matter.

The radiation so produced is released as two photons travelling in near opposite directions. Hence, by detecting corresponding photons within a small co-incidence timing window (an event), a line of response (LOR) along which the origin of the radiation lies can be deduced. Deduction (or analysis) of many such lines allows a three-dimensional map of the tracer distribution to be produced that maps onto the spatial dimensions of the subject. The number of individual events emitted and hence, detected, is related to the rate of radioactive decay and hence, the concentration of radioactive tracer in the subject. Confidence in the deduced radioactivity distribution from the lines of response is proportional to the number of events detected which is related in turn to the time interval over which the events are recorded. Some tracers, such as FDG, are known to accumulate in organs that have high metabolic rate of glucose such as tumor cells and muscles. In such cases, events are recorded for an interval of time after the tracer has had time to accumulate and an average rate of decay is estimated for that interval. Such images are known as static scans.

Alternatively, several such images can be produced over a certain period of time while the tracer distribution is still evolving to form a sequence of temporal frames analogous to a video recording of the functional activity as labelled by the radiotracer. These sequences are conventionally called dynamic images and can be 2 or 3 dimensional.

Analysis of how the radiotracer distributes over time as well as space provides additional important information about the functional behaviour of the subject over and above that attainable by static images.

The analysis of these dynamic images concerning contrast agent or radioactive tracer uptake frequently involves pharmacokinetic (PK) modelling techniques in which the cellular process of molecule intake and wash-out is modelled as a diffusion process in multiple, well defined compartments (see for example Positron Emission Tomography Compartmental Models; R. N. Gunn, S. R. Gunn and V. J. Cunningham; Journal of Cerebral Blood Flow and Metabolism, vol. 21, no. 6, pp. 635-652, 2001).

The pharmacokinetic approach is fundamental to modelling drug action on tissues of interest. It is routinely utilized in drug development techniques as the medical imaging modalities allow the visualization of some in vivo cellular processes.

The resolution of the PK model involves fitting of parameters of the model to the temporal image data. The mathematical solution of the relative concentration of agent in tissue is a convolution of two functions:
a sum of decreasing exponential functions of time, characterizing the wash-in and wash-out of agents in each compartment;
a time varying function proportional to the concentration of tracer in the blood vessels, i.e. the Blood Input Function (BIF).

In order to be accurate and truly quantitative, PK modelling techniques require a reasonably accurate determination of the BIF.

A number of approaches to the estimation of BIF are known and each of these has associated disadvantages.

In one approach, a region of interest (ROI) is drawn in the area of an artery on a PET image and the uptake in the ROI is assumed to be linearly related to the BIF. Since the useable arteries are small relative to the attainable resolution of the imaging device, (no more than a few mm or voxels), the estimation is inaccurate as it is very sensitive to the placement of the ROI and to noise. Typically, the spatial resolution of PET does not allow estimation of an accurate BIF in such small ROIs. If the ROI is smaller than 1 cm in diameter, the quantification in the RIO becomes problematic since it is affected by high variance or bias in the estimates. Moreover, the temporal resolution of the dynamic image is compromised with the noise level: the shorter the frame rate, the more noisy the image. For example, in some cases, if the frame lasts for less than 5-10 seconds, the noise in the image is such that quantification also becomes inaccurate.

A more common approach involves drawing serial blood samples over time and then counting a measured volume of blood from each sample in a radiation detector. This is an invasive, traumatic process which involves risks to the subject and practitioner alike and is complicated by the need to handle radioactive blood. In addition to the risks to personnel associated with contamination, there is the risk that complex, expensive equipment could be rendered inoperable while the necessary decontamination is carried out.

For animal studies (particularly murine) it should be noted that the amount of blood needed to estimate the BIF may be a significant fraction of the total amount of blood in the animal and frequent blood sampling can lead to the death of the animal through excessive blood loss. This generally prohibits use of the same animal for successive studies, a factor which radically changes experimental set-up and protocol, as well as substantially increasing the cost of performing such studies since many more animals are required for the experiment.

For further discussion of the issues arising from the different approaches to estimation of BIF currently used, see for example Parametrically defined cerebral blood vessels as non-invasive blood input functions for brain PET studies; M Asselin, V J Cunningham, Shigeko Amano, R N Gunn and C Nahmias; Phys. Med. Biol. 49 (2004) 1033-1054 and Measurement of input functions in rodents: challenges and solutions; R Laforest, et al/Nuclear Medicine and Biology 32 (2005) 679-685.

PET is associated with at least two major advantages. First, the radionuclides used (C, N, F etc) can be bound to almost any organic molecule and hence potentially to a great number of molecules which would be involved in a given metabolic pathway. This makes imaging of almost any cellular process possible.

PET is also extremely sensitive and only very small amounts of tracer are necessary to obtain a useful signal (typically nM or pM concentrations are sufficient to get usable signal).

On the other hand, PET does suffer from one major drawback: if an image of useable signal to noise ratio is to be produced, then the temporal resolution is very poor. Typically, a PET image acquired in 10-30 seconds will yield a noisy image with low signal to noise ratio (SNR).

This is the main reason why PET is not routinely used in clinical practice as a dynamic imager: only molecular radioactive tracers which have a short wash-out time and well understood pharmaco-dynamic properties are imaged in PET clinical routines, as a static image after the tracer uptake has reached a steady state.

Dynamic PET is only usually used in research protocols and the accurate estimation of BIF is the subject of intense research.

If good SNR and/or quantitative information were available from PET or other functional medical scanning techniques (e.g. SPECT) then the utility of the modality would be greatly extended, particularly in preclinical work and subsequently in the clinical domain. In SPECT, possible contrast agents include 99m-Technetium-MIBI or SESTA-MIBI.

Magnetic Resonance Imaging (MRI) is a technique having a set of advantages and disadvantages that are complementary to those of PET.

It is a very fast modality in which 2D images of SNR around 5 or 10 can be acquired in real time (e.g. 20 images per second). Consequently, the use of T1-based contrast agents like Gadolinium (Gd) chelates has been generalised in clinical practice, such as dynamic contrast enhanced MRI (DCE-MRI) of the breast, to great effect: regions of fast uptake of the Gd agent can be identified and characterized as malignant (distinguished from regions of slower uptake) with good specificity. Although classic T1 contrast agents comprise Gd chelates, others may be used.

Anatomy is also clearly visible in an MRI image so identification of arterial tissue is easier. The combination of high spatial resolution which provides good anatomical detail, together with the high temporal resolution, provides an unparalleled ability to track the Gd molecules in the blood stream and this can be used to calculate a BIF. For example, see Magn Reson Med. 1996 August; 36 (2): 225-31, Measurement of the arterial concentration of Gd-DTPA using MRI: a step toward quantitative perfusion imaging. Fritz-Hansen T, Rostrup E, Larsson H B, Sondergaard L, Ring P, Henriksen O.

On the other hand, the sensitivity is lower than that of PET and agent concentrations of up to mM or micro-M are required in order to obtain a useable signal.

Also, the T1 contrast agents with Gd chelates are large molecules which do not penetrate the cell. Metabolic pathways that can be imaged using DCE MRI are restricted to those involving intracellular station or cellular membrane receptor binding of the agent. This places significant limitation on MRI as a tool for disease assessment and exploration of drug delivery because most metabolic processes occur within the cell.

According to the invention, a method of processing a set of functional medical scan images comprises the steps set out in claim 1 attached hereto.

In addition to data acquired from the MRI scan, a prior model, or data acquired during the functional medical scan, may be used to calculate the BIF.

The functional medical scan images could be, for example PET scan images of SPECT scan images.

The MRI contrast agent and the functional medical scan contrast agent are conveniently injected as a mixture.

The invention will now be described, by way of non-limiting example, with reference to the following figures in which:

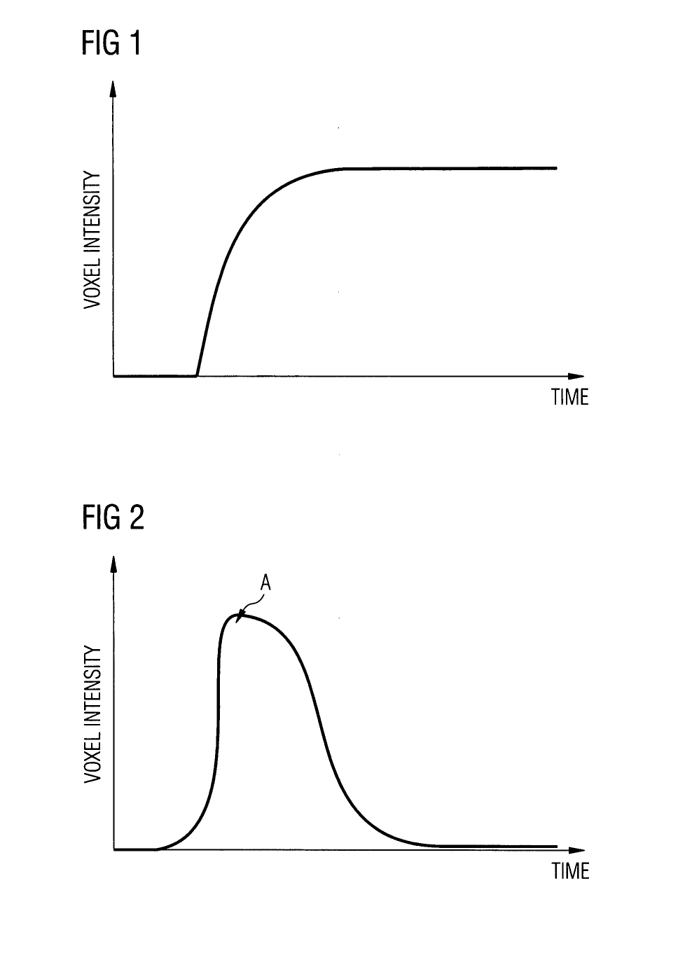

FIG. 1 is a schematic sketch of the kinetic concentration curve for concentration of radioactive tracer in tissue following injection of the tracer in a subject and

FIG. 2 a schematic sketch of the kinetic concentration curve for concentration of radioactive tracer in plasma following injection of the tracer in a subject (BIF).

FIGS. 1 and 2 illustrate the functions which need to be characterised in a subject in order to apply pharmacokinetic modelling techniques to the data acquired from functional medical scanning techniques such as PET. The data necessary to derive a real function such as illustrated in FIG. 1 would typically come from regional analysis of a sequence of PET images obtained over scan intervals of varying lengths. For a real function such as illustrated in FIG. 2, the data might typically be provided by plasma radioactivity concentration in well counted blood samples drawn over time.

From the shape of the BIF illustrated in FIG. 2, and in particular the relatively sharp and high peak A, it can be seen that the method of acquiring data needs to have a relatively high temporal resolution in order to define the function accurately.

By one embodiment of the current invention, a living object is simultaneously imaged using MRI and PET. The contrast agent used is a combination of Gd chelate (or any other T1 contrast agent) and a PET radionuclide. Such a combination could be administered as separate injections of the T1 agent and the radionuclide or a single injection of a mixture of the two.

Alternatively, the functional group necessary for each of the two modalities could be incorporated in a single molecule.

An MRI image is acquired in an area where the arteries or blood pool (cardiac ventricles) of the living object are easy to identify and the fast imaging possible enables accurate estimation of the BIF. A high concentration of Gd chelate can be used to obtain a good signal.

A PET image is acquired using the other agent which binds to the desired receptor. The slow process of uptake in the tissue is adequately imaged in PET.

The PET signal can be subsequently analysed using PK modelling techniques and a reliable estimation of the BIF obtained from the MRI data.

Alternatively the BIF may be calculated from a combination of the MRI data and a prior model of the BIF. which may be derived from previous studies, prior knowledge or population norms. Data from the functional medial scan could also be combined with the MRI data to calculate the BIF.

The fields of view (FOV) for the PET and MRI scans need not be overlapping as the arterial signal can be usefully obtained in any part of the body. Thus, an arrangement is possible where the PET and MRI apparatus are discrete equipments that are located close together.

However, use of a combined PET-MRI system (see Ladebeck et al., ISRMR 2005) can provide for PET and MRI scans that cover the same field of view. A crucial advantage of the device resides in the possibility of acquiring the data simultaneously; this element is essential as the distribution of the tracer in the blood can hardly be reproduced through successive injections.

A common anatomical framework is thus established in which the results of scans in the two modalities can be combined (even when adjustment is required for e.g. breathing). The combination of MRI and PET establishes a common spatiotemporal frame within which the dynamic information from each can be combined.

Such a combined system also lends itself to administration of the PET and MRI contrast agents in a single injection.

Since it is not necessary to have equal amounts of MR agent and radionuclide, adequate signal can be obtained from standard PET doses. If the MR agent is used simply to trace blood pool and permeability, a conventional Gd-chelate can be used. If the MR agent needs to be more specific, Gd could be accumulated on to nano-tubes on which a suitable PET radionuclide is also bound. It is possible to accumulate up to tens of thousands of Gd on a single nano-tube, thereby increasing the sensitivity of MRI.

BIF can also be obtained using alternatives to injectable contrast agents e.g. by using arterial spin labelling or from the haemodynamic response as used in functional MRI (fMRI).

1. A method of processing a set of quantitative functional medical scan images comprising the steps of:
administering a suitable functional medical scan contrast agent and a suitable MRI contrast agent to a subject;
simultaneously performing a functional medical scan and a MRI scan on the subject to generate the functional medical scan images and corresponding data from the MRI scan;
calculating the blood input function (BIF) for the subject using the data acquired during the MRI scan and
performing pharmacokinetic analysis based on the BIF so derived and the quantitative functional medical scan images.

2. A method according to claim 1 where the BIF is calculated using a prior model of the BIF in addition to the MRI data.

3. A method according to claim 1 where the BIF is calculated using the functional medical scan images in addition to the MRI data.

4. A method according to claim 1, where the functional medical scan and the MRI scan are performed on overlapping fields of view on the subject.

5. A method according to claim 1, where the functional medical scan is a PET scan.

6. A method according to claim 1, where the functional medical scan is a SPECT scan.

7. A method according to claim 1, where the functional medical scan contrast agent and the MRI contrast agent are injected as a mixture.
